(19) United States
(12) Patent Application Publication
(10) Pub. No.: US 2023/0372334 A1
(43) Pub. Date: Nov. 23, 2023

(54) USE OF AN ERK INHIBITOR FOR THE TREATMENT OF MYELOFIBROSIS

(71) Applicants: Novartis AG, Basel (CH); University of Basel, Basel (CH)

(72) Inventors: Sime BRKIC, Basel (CH); Hans MENSSEN, Riehen (CH); Sara MEYER, Liestal (CH); Thomas RADIMERSKI, Augst (CH); Simona STIVALA, Liestal (CH)

(21) Appl. No.: 18/248,119

(22) PCT Filed: Oct. 7, 2021
(86) PCT No.: PCT/IB2021/059202

§ 371 Date: Apr. 6, 2023

Related U.S. Application Data
(60) Provisional application No. 63/089,168, filed on Oct. 8, 2020.

(51) Int. Cl. A61K 31/4965; A61K 31/519; A61P 35/00

(52) U.S. Cl. CPC A61K 31/4965; A61K 31/519; A61P 35/00

(57) ABSTRACT

The invention relates to the use of an ERK inhibitor in the treatment of myelofibrosis (MF).

### FIELD OF THE INVENTION

The present invention provides uses of ERK inhibitors in the treatment of a disease or disorder as described herein, or methods of treating a disease or disorder as described herein.

The present invention provides an ERK inhibitor for use in treating myeloproliferative neoplasms (MPNs), including the treatment of myelofibrosis (MF), essential thrombocythemia (ET) and/or polycythemia vera (PV). The present invention relates to use of an ERK inhibitor for the treatment of myelofibrosis (MF).

The present invention provides the use of an ERK inhibitor, e.g. 4-(3-amino-6-((1S,3S,4S)-3-fluoro-4-hydroxycyclohexyl)pyrazin-2-yl)-N-((S)-1-(3-bromo-5-fluorophenyl)-2-(methylamino)ethyl)-2-fluorobenzamide (Compound A), in the treatment of myeloproliferative neoplasms (MPNs), including the treatment of myelofibrosis (MF), essential thrombocythemia (ET) and/or polycythemia vera (PV).

The present invention provides the use of an ERK inhibitor, e.g. 4-(3-amino-6-((1S,3S,4S)-3-fluoro-4-hydroxycyclohexyl)pyrazin-2-yl)-N-((S)-1-(3-bromo-5-fluorophenyl)-2-(methylamino)ethyl)-2-fluorobenzamide (Compound A), in the treatment of myeloproliferative neoplasms (MPNs), including the treatment of myelofibrosis (MF), essential thrombocythemia (ET) and/or polycythemia vera (PV). The invention also provides a pharmaceutical composition for the treatment of MF comprising an ERK inhibitor and optionally a pharmaceutically acceptable excipient.

The invention also provides a method of treating a disease or disorder, in particular myeloproliferative neoplasms (MPNs), including the treatment of myelofibrosis (MF), essential thrombocythemia (ET) and/or polycythemia vera (PV), in a patient in need thereof comprising administering to said patient a jointly therapeutically effective amount of an ERK inhibitor or a pharmaceutical composition comprising an ERK inhibitor. Also provided are uses of the pharmaceutical compositions for the treatment of a disease or disorder, in particular myeloproliferative neoplasms (MPNs), including the treatment of myelofibrosis (MF), essential thrombocythemia (ET) and/or polycythemia vera (PV). The present invention also provides pharmaceutical compositions comprising an ERK inhibitor and commercial packages thereto, and their uses in treating a disease or a disorder as described herein.

### BACKGROUND OF THE INVENTION

Myeloproliferative neoplasms (MPNs) are a unique and heterogeneous group of hemopathies characterized by proliferation and accumulation of mature myeloid cells. MPNs, include myelofibrosis (MF), essential thrombocythemia (ET) and polycythemia vera (PV). Importantly, MF is the most severe form of Philadelphia chromosome-negative (i.e. BCR-ABL1-negative) myeloproliferative neoplasms, with a prevalence estimated to be 2.2 per 100,000 population. MF can present as a de novo disorder as a primary hematologic malignancy, primary myelofibrosis (PMF) or evolve from previous myeloproliferative neoplasms, namely: PV, post-PV MF (PPV-MF), ET, or post-ET MF (PET-MF). The range of reported frequencies for post-PV MF are 4.9-6% at 10 years and 6-14% at 15 years, respectively, and 0.8-4.9% for post-ET MF at 10 years and 4-11% at 15 years, respectively (S Cerquozzi and A Tefferi, Blood Cancer Journal (2015) 5, e366).

Regardless of whether MF developed from PV, ET or as a primary disorder, it is characterized by a clonal stem cell proliferation associated with production of elevated levels of several inflammatory and proangiogenic cytokines resulting in a bone marrow stromal reaction that includes varying degrees of reticulin and/or collagen fibrosis, osteosclerosis and angiogenesis, some degree of megakaryocyte atypia and a peripheral blood smear showing a leukoerythroblastic pattern with varying degrees of circulating progenitor cells. The abnormal bone marrow milieu results in release of hematopoietic stem cells into the blood, extramedullary hematopoiesis, and organomegaly at these sites. Clinically, MF is characterized by progressive anemia, leukopenia or leukocytosis, thrombocytopenia or thrombocythemia and multi-organ extramedullary hematopoiesis, which most prominently involves the spleen leading to massive splenomegaly, severe constitutional symptoms, a hypermetabolic state, cachexia, and premature death.

A considerable number of cytokine and growth factor receptors utilize non-receptor tyrosine kinases, the Janus kinases (JAK), to transmit extracellular ligand binding into an intracellular response. For example, erythropoietin, thrombopoietin and granulocyte monocyte colony stimulating factor are all known to signal through receptors that utilize JAK2. JAK activate a number of downstream pathways implicated in proliferation and survival, including the STATs (signal transducers and activators of transcription), a family of important latent transcription factors.

Myelofibrosis is now known to be a clonal stem cell disease characterized by molecular (JAK2V617F, MPLW515L1K) and cytogenetic (13q-,20q-) markers (Pikman Y, Lee B H, Mercher T, et al. PLoS Med. 2006;3(7):e270; Scott L M, Tong W, Levine R L, et al. N Engl J Med. 2007; 356:459-468). The JAK2V617F mutation has been identified in over 95% of patients with PV and approximately 50% of patients with ET and PMF. Furthermore, in a preclinical setting, animal studies have demonstrated that this mutation can lead to an MF-like syndrome. The JAK2V617F mutation alters the JAK2 tyrosine kinase making it constitutively active. As a result, polycythemia, thrombocythemia and leukocytosis can develop independently from growth factor regulation. Even in patients lacking a confirmed JAK2 mutation, the detection of STAT activation suggests dysregulated JAK activity. In fact, regardless of the mutational status of JAK2, the malignant cells appear to retain their responsiveness to JAK activating cytokines and/or growth factors; hence, they may benefit from JAK inhibition. Although several JAK inhibitors, including ruxolitinib (brand name Jakavi) have been approved for the treatment of MF, they have only demonstrated an effect in the treatment of symptoms. Progression of the disease is not halted and eventually patients may die prematurely.

Patients with MF have shortened survival (median survival is 6.5 years) and greatly compromised quality of life (QoL). Contributing factors for shortened survival include leukemic transformation and thrombohemorrhagic complications and for the compromised quality of life severe anemia (often requiring red blood cell (RBC) transfusions), symptomatic enlargement of the spleen and liver, substantial MF-associated symptoms burden (MF-SB), and cachexia (Tefferi and Barbui 2019).

The only potential curative treatment for MF is allogeneic hematopoietic stem cell transplantation (ASCT), for which the great majority of patients are ineligible. Therefore, treatment options remain primarily palliative and aimed at controlling disease symptoms, complications and improving the patient's QoL. The therapeutic landscape of MF has changed with the discovery of the V617F mutation of the Janus kinase JAK2 gene present in 60% of patients with PMF or PET-MF and in 95% of patients with PPV-MF, triggering the development of molecular targeted therapy for MF (Cervantes 2014). JAK play an important role in signal transduction following cytokine and growth factor binding to their receptors. Aberrant activation of JAK has been associated with increased malignant cell proliferation and survival (Valentino and Pierre 2006). JAK activate a number of downstream signaling pathways implicated in the proliferation and survival of malignant cells including members of the Signal Transducer and Activator of Transcriptions (STAT) family of transcription factors.

JAK inhibitors were developed to target JAK2 thereby inhibiting JAK signaling. Ruxolitinib, as all agents of this class, mainly inhibits dysregulated JAK-STAT signaling present in all MF patients irrespective of their JAK2 mutational status, but is not selective for the mutated JAK2, which explains its efficacy in both JAK2-positive and -negative MF. Ruxolitinib is highly effective in reducing the spleen size and controlling the symptoms of MF, with this resulting in a marked improvement in the patient's QoL (Cervantes et al 2016). Ruxolitinib is the only JAK inhibitor that has been granted a marketing authorization, as a single agent, for the treatment of patients with PMF, PPV-MF or PET-MF and for the treatment of patients with PV who are resistant to or intolerant to hydroxyurea. Ruxolitinib is the only approved pharmacological treatment for MF patients with splenomegaly and/or clinical symptoms and is considered standard of care (SoC). Although ruxolitinib has changed the treatment paradigm of MF patients, there is no clear indication of its disease-modifying effect (Cervantes 2014) and therapy-related anemia is often an anticipated downside (Naymagon and Mascarenhas 2017, Mead et al 2015). While ruxolitinib demonstrates improvements in splenomegaly and constitutional symptoms, it has not been shown to improve anemia.

Current treatment options post JAK inhibitors are limited in their efficacy, durability and tolerability. Multiple efforts are currently ongoing to improve the outcome of patients with MF post JAK inhibitors identifying new agents or combinations, such as those targeting cellular metabolic and apoptotic pathways, cell cycle and immune therapy. There remains a high unmet medical need to finding new and efficacious therapeutic options for advancing the treatment of MF. There is also a need for targeted therapy that is safe and/or well tolerated. For example, there is a need for a therapy that would help to overcome the side effects such as anemia, which are associated with standard of care such as monotherapy with ruxolitinib.

WO/2015/066188 describes ERK 1/2 inhibitors such as Compound A, also known as rineterkib, as being useful in treating diseases such as cancer that are associated with excessive activity of ERK1 and/or ERK2. However, it does not specifically disclose the use of such ERK1/2 inhibitors in the treatment of myeloproliferative neoplasms such as MF, ET, and PV.

### SUMMARY OF THE INVENTION

Inhibition of JAK2 and ERK1/2 with a combination of ruxolitinib and several ERK inhibitors (such as Compound A and MK-8535) inhibited proliferation of Jak2V617F Ba/F3 cells. Treatment of mice competitively transplanted with Jak2V617F and wild-type BM with the Compound A in combination with a JAK2 inhibitor corrected erythrocytosis and splenomegaly. Longer-term treatment was able to induce clone reductions. BM fibrosis was profoundly decreased in MPLW515L-driven MPN to an extent not seen with JAK2 inhibitor monotherapy. Myeloid colony formation from JAK2V617F patients' CD34+blood and BM was dose-dependently inhibited by combined JAK2/ERK1/2 inhibition in PV, ET and MF subsets. The fitness of the MPN clone was found to be decreased.

It was found that an ERK 1/2 inhibitor such as Compound A as defined below significantly normalizes splenomegaly and polycythemias and reduces elevated hematocrit in a MF mouse model. The ERK 1/2 inhibitor (Compound A) was also found to be well tolerated in a MF mouse model.

The present invention therefore provides a novel therapy which may deliver clinical benefit to a patient suffering from MPNs such as MF and/or PV. In particular, the present invention may provide an improvement of anemia and progression free survival for such patients.

The present invention thus provides methods, or compounds for use in the treatment of a disorder or disease or for use in the alleviation of a symptom or symptoms associated with the disorder or disease as described herein.

In an embodiment, the ERK1/2 inhibitor is selected from Compound A (rineterkib), BVD-523 (ulixertinib), GDC-0994, KO-947, Vtx-11e, SCH-772984, MK2853, LY3214996, BVD-523, SCH-722984, LY3214996, SCH-900353, AEZS-140, AEZS-131, AEZS-136, RG-7842 CC-90003, KIN-4050, and combinations thereof.

In an embodiment, the ERK1/2 inhibitor is Compound A (rineterkib), BVD-523 (ulixertinib), SCH-772984, MK2853, SCH-722984, or DEL22379.

In an embodiment, the ERK1/2 inhibitor is Compound A (rineterkib), SCH-772984, MK2853, or SCH-722984.

The present invention thus provides a medicament for the treatment of myelofibrosis. The present invention is based on the inventors' surprising finding that an ERK1/2 inhibitor is useful in the treatment of myelofibrosis in a patient.

In an embodiment, the ERK1/2 inhibitor is a compound having the structure of Formula (I)

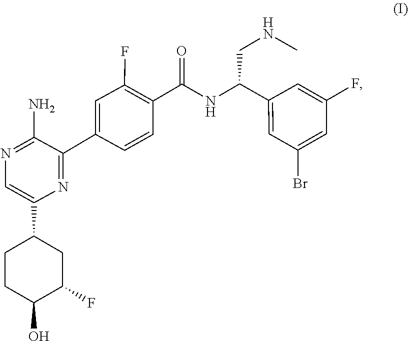 [SMILES: CNC[C@@H](NC(=O)c1ccc(-c2nc([C@H]3CC[C@H](O)[C@@H](F)C3)cnc2N)cc1F)c1cc(F)cc(Br)c1]

4-(3-amino-6-((1S,3S,4S)-3-fluoro-4-hydroxycyclohexyl)pyrazin-2-yl)-N-((S)-1-(3-bromo-5-fluorophenyl)-2-(methylamino)ethyl)-2-fluorobenzamide (“Compound A”) or a pharmaceutically acceptable salt thereof, for example the hydrochloride salt thereof.

### BRIEF DESCRIPTION OF THE DRAWINGS

FIG. 1A depicts the antiproliferative effect of Compound A (LTT462) and ruxolitinib (Rux) in the Ba/F3 EpoR JAK2V617F cell line.

FIG. 1B depicts the antiproliferative effect of Compound A (LTT462) and ruxolitinib (Rux) in the SET2 cell line.

FIG. 2A depicts the effects of vehicle, ruxolitinib (Rux) and Compound A (LTT462) on ERK signaling in JAK2V617F PV/MF mouse model.

FIG. 2B depicts the effects of vehicle, ruxolitinib (Rux) and Compound A (LTT462) on hematocrit levels in JAK2V617F PV/MF mouse model.

FIG. 2C depicts the effects of vehicle, ruxolitinib (Rux) and Compound A (LTT462) on spleen weight in JAK2V617F PV/MF mouse model.

FIG. 2D depicts the effects of vehicle, ruxolitinib (Rux) and Compound A (LTT462) on white blood count WBC count in in MPLW515L mice.

FIG. 3A depicts the activity of vehicle, ruxolitinib (Rux), and Compound A (LTT462), on the reduction of elevated hematocrit in JAK2V617F PV/MF mouse model.

FIG. 3B depicts the activity of vehicle, ruxolitinib (Rux), and Compound A (LTT462), on normalizing splenomegaly in JAK2V617F PV/MF mouse model.

FIG. 4A depicts the tolerability of the treatments with vehicle, ruxolitinib (Rux), and Compound A (LTT462), as indicated by body weight change over time.

FIG. 4B depicts the tolerability of the treatments with vehicle, ruxolitinib (Rux), and Compound A (LTT462), as indicated by bone marrow cellularity, indicating absence of bone marrow toxicity.

FIG. 4C depicts the tolerability of the treatments with vehicle, ruxolitinib (Rux), and Compound A (LTT462), as indicated by normal WBC count.

FIG. 4D depicts the tolerability of the treatments with vehicle, ruxolitinib (Rux), and Compound A (LTT462), as indicated by normal platelet count, indicating absence of thrombocytopenia.

FIG. 5A depicts in vitro activity of vehicle, Compound A (LTT462), and ruxolitinib (Rux) in CD34+peripheral blood mononuclear cells (PBMCs).

FIG. 5B depicts the activity of vehicle (Veh), Compound A (LTT462), and ruxolitinib (Rux) on hematocrit levels in JAK2V617F mice.

FIG. 6 depicts the effects of Compound A (LTT462), and ruxolitinib (Rux) on IC50 activity in EpoR Jak2V617F mutant and Jak2 wild type Ba/F3 cells.

### DETAILED DESCRIPTION OF THE INVENTION

Certain terms used herein are described below. Compounds or biological agents of the present invention are described using standard nomenclature. Unless defined otherwise, all technical and scientific terms used herein have the same meaning as is commonly understood by one of skill in the art to which this invention belongs.

The term “JAK inhibitor” as used herein refers to a compound that selectively targets, decreases, or inhibits at least one activity of JAK.

The term “JAK1/2 inhibitor” as used herein refers to a compound that selectively targets, decreases, or inhibits the JAK 1 and JAK 2 tyrosine kinases.

The term “ERK inhibitor” as used herein refers to a compound that inhibits extracellular signal-regulated kinase (ERK).

The term “ERK 1/2 inhibitor” as used herein refers to a compound that inhibits ERK1 and/or ERK2 kinases.

The term “pharmaceutical composition” is defined herein to refer to a mixture or solution containing at least one therapeutic agent to be administered to a patient, e.g., a mammal or human, in order to prevent or treat a particular disease or condition affecting the mammal.

The term “pharmaceutically acceptable” as used herein refers to those compounds, biological agents (e.g., antibodies), materials, compositions and/or dosage forms, which are, within the scope of sound medical judgment, suitable for contact with the tissues of a warm-blooded animal, e.g., a mammal or human, without excessive toxicity, irritation, allergic response, and other problem complications commensurate with a reasonable benefit/risk ratio.

The terms “fixed dose” and “single formulation” as used herein refers to a single carrier or vehicle or dosage form formulated to deliver an amount, which is jointly therapeutically effective for the treatment or prevention of cancer, of both therapeutic agents to a patient. The single vehicle is designed to deliver an amount of the agent, along with any pharmaceutically acceptable carriers or excipients. In some embodiments, the vehicle is a tablet, capsule, pill, or a patch. In other embodiments, the vehicle is a solution or a suspension.

An “oral dosage form” includes a unit dosage form prescribed or intended for oral administration.

The term “treating” or “treatment” as used herein comprises a treatment relieving, reducing, or alleviating at least one symptom in a patient or effecting a delay of progression of a disease. For example, treatment can be the diminishment of one or several symptoms of a disorder or complete eradication of a disorder, such as cancer. Within the meaning of the present disclosure, the term “treat” also denotes to arrest, delay the onset (i.e., the period prior to clinical manifestation of a disease), and/or reduce the risk of developing or worsening a disease. The term “protect” is used herein to mean prevent, delay, or treat, or all, as appropriate, development, continuance or aggravation of a disease in a patient, e.g., a mammal or human. The term “prevent”, “preventing” or “prevention” as used herein comprises the prevention of at least one symptom associated with or caused by the state, disease or disorder being prevented.

The term “treatment” as used herein includes treatment of splenomegaly, treatment of hepatomegaly, treatment of thrombocytopenia, treatment of neutropenia, treatment of anemia, treatment of bone marrow fibrosis associated with MF, and treatment of a symptom associated with MPNs or a constitutional symptom associated with myelofibrosis.

The term “pharmaceutically effective amount,” “therapeutically effective amount,” or “clinically effective amount” of therapeutic agents is an amount sufficient to provide an observable or clinically significant improvement over the baseline clinically observable signs and symptoms of the disorders treated with the therapeutic agent.

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, biological agents, salts, and the like, this is taken to mean also a single compound, salt, or the like.

The terms “about” or “approximately” are generally understood by persons knowledgeable in the relevant subject area, but in certain circumstances can mean within 20%, within 10%, or within 5% of a given value or range. Alternatively, especially in biological systems, the term “about” means within about a log (i.e., an order of magnitude) or within a factor of two of a given value.

In particular, where a dosage is mentioned as ‘about’ a particular value, or a particular value (i.e. without the term “about” preceding that particular value, it is intended to include a range around the specified value of plus or minus 10%, or plus or minus 5%. As is customary in the art, dosages refer to the amount of the therapeutic agent in its free form. For example, when a dosage of 100 mg of Compound A is referred to, and Compound A is used as its hydrochloride salt, the amount of the therapeutic agent used is equivalent to 100 mg of the free form of Compound A.

In an embodiment, the ERK1/2 inhibitor is selected from Compound A (rineterkib), BVD-523 (ulixertinib), GDC-0994, KO-947, Vtx-11e, SCH-772984, MK2853, LY3214996, BVD-523, SCH-722984, LY3214996, SCH-900353, AEZS-140, AEZS-131, AEZS-136, RG-7842 CC-90003, KIN-4050, and combinations thereof.

In an embodiment, the ERK1/2 inhibitor is Compound A (rineterkib), BVD-523 (ulixertinib), SCH-772984, MK2853, SCH-722984, or DEL22379.

In an embodiment, the ERK1/2 inhibitor is Compound A (rineterkib), SCH-772984, MK2853, or SCH-722984.

In one preferred embodiment, the ERK1/2 inhibitor is Compound A, which is 4-(3-amino-6-((1S,3S,4S)-3-fluoro-4-hydroxycyclohexyl)pyrazin-2-yl)-N-((S)-1-(3-bromo-5-fluorophenyl)-2-(methylamino)ethyl)-2-fluorobenzamide:

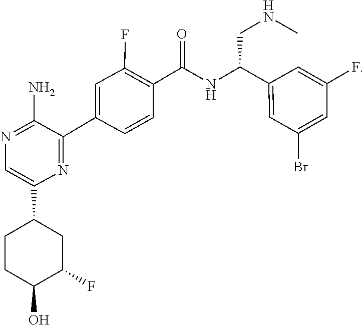 [SMILES: CNC[C@@H](NC(=O)c1ccc(-c2nc([C@H]3CC[C@H](O)[C@@H](F)C3)cnc2N)cc1F)c1cc(F)cc(Br)c1]

This compound is an inhibitor of ERK 1 and ERK 2. The compound is disclosed and its preparation described in published PCT patent application WO2015/066188 as example 184, which is incorporated herein by reference. This compound is also known as rineterkib. In some embodiments, this compound is used as its hydrochloride salt.

Any reference to “Compound A” herein is meant to include a reference to Compound A, or a pharmaceutically acceptable salt thereof, for example the hydrochloride salt thereof, unless context clearly indicates otherwise.

The present invention provides uses of ERK inhibitors and combinations thereof, and their uses in the treatment of a disease or disorder as described herein, or methods of treating a disease or disorder as described herein.

In one embodiment, an ERK inhibitor that can be used in the embodiments of the invention is BVD-523, also known as ulixertinib, which is (S)-4-(5-chloro-2-(isopropylamino)pyridin-4-yl)-N-(1-(3-chlorophenyl)-2-hydroxyethyl)-1H-pyrrole-2-carboxamide:

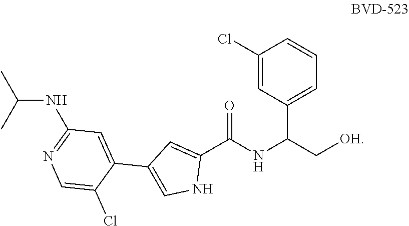 [SMILES: CC(C)Nc1cc(-c2c[nH]c(C(=O)NC(CO)c3cccc(Cl)c3)c2)c(Cl)cn1]

Exemplary JAK inhibitors include, but are not limited to, ruxolitinib (Jakafi®); tofacitinib (CP690550); axitinib (AG013736, CAS 319460-85-0); 5-Chloro-N2-[(1S)-1-(5-fluoro-2-pyrimidinyl)ethyl]-N4-(5-methyl-1H-pyrazol-3-y)-12,4-pyrimidinediamine (AZD1480, CAS 935666-88-9); (9E)-15-[2-(1-Pyrrolidinyl)ethoxy]-7,12,26-trioxa-19,21,24-triazatetracyclo[18.3.1.12,5.114,18]-hexacosa-1(24),2,4,9,14,16,18(25),20,22-nonaene (SB-1578, CAS 937273-04-6); momelotinib (CYT 387); baricitinib (INCB-028050 or LY-3009104); pacritinib (SBI 518); (16E)-14-Methyl-20-oxa-5,7,14,27-tetraazatetracyclo[19.3.1.12,6.18,12]heptacosa-1(25),2,4,6(27),8,10,12(26),16,21,23-decaene (SB 1317); gandotinib (LY 2784544); and N,N-cicyclopropyl-4-[(1,5-dimethyl-1H-pyrazol-3-yl)amino]-6-ethyl-1,6-dihydro-1-methyl-imidazo[4,5-d]pyrrolo[2,3-b]pyridine-7-carboxamide (BMS 911543).

As used herein, “ruxolitinib” is the JAK1/JAK2 inhibitor (R)-3-(4-(7H-pyrrolo[2,3-d]pyrimidin-4-yl)-1H-pyrazol-1-yl)-3-cyclopentylpropanenitrile, also named 3(R)-Cyclopentyl-3-[4-(7H-pyrrolo[2,3-d]pyrimidin-4-yl)-1H-pyrazol-1-yl]propanenitrile, of formula:

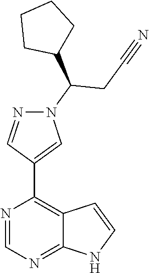 [SMILES: N#CC[C@H](C1CCCC1)n1cc(-c2ncnc3[nH]ccc23)cn1]

which can be prepared, for example, as described in WO2007/070514, which is incorporated herein by reference. As used herein, “ruxolitinib” refers to the free form, and any reference to “a pharmaceutically acceptable salt thereof” refers to “a pharmaceutically acceptable acid addition salt thereof”, in particular ruxolitinib phosphate, which can be prepared, for example, as described in WO2008/157208, which is incorporated herein by reference. Ruxolitinib is approved for the treatment of intermediate to high-risk myelofibrosis under the tradename Jakafi®/Jakavi®.

As used herein, “ruxolitinib” is also intended to represent ruxolitinib, or a pharmaceutically acceptable salt thereof (for example the phosphate salt thereof), unless context clearly indicates otherwise.

Treatment of Myeloproliferative Neoplasms (MPNs) and of Myelofibrosis

Myeloproliferative neoplasms (MPNs) are hematopoietic stem cell disorders characterized by excessive output of mature myeloid blood cells and an inherent risk for transformation to acute myeloid leukemia. MPN subtypes include; polycythemia vera (PV) primarly with polyglobulia, essential thrombocythemia (ET) with thrombocytosis, and myelofibrosis (MF) with an initial cell-rich phase followed by progressive bone marrow (BM) fibrosis and cytopenias. All of these MPN subsets share a common feature of dysregulated JAK2 signaling3, which is constitutively activated by somatic mutations in JAK2, the thrombopoietin receptor MPL or its stabilizing chaperone calreticulin (CALR)4.

Reference to the treatment of myeloproliferative neoplasms (MPNs) throughout this specification is therefore intended to include a disease or disorder selected from myelofibrosis (MF), essential thrombocythemia (ET), polycythemia vera (PV), and combinations thereof. Fpr example, the term “treatment” includes treatment of polycythemia vera (PV) primarly with polyglobulia, essential thrombocythemia (ET) with thrombocytosis, and myelofibrosis (MF) with an initial cell-rich phase followed by progressive bone marrow (BM) fibrosis and cytopenias.

In one aspect, the present invention provides an ERK1/2 inhibitor (e.g., Compound A) or a pharmaceutical acceptable salt thereof, for use in the treatment of myeloproliferative neoplasms (MPNs).

In one aspect, the present invention provides an ERK1/2 inhibitor (e.g., Compound A) or a pharmaceutical acceptable salt thereof, for use in the treatment of (i) myelofibrosis (MF), (ii) essential thrombocythemia (ET) or (iii) polycythemia vera (PV).

Myelofibrosis comprises primary myelofibrosis (PMF), post-essential thrombocythemia myelofibrosis (PET-MF) and post-polycythemia vera myelofibrosis (PPV-MF). Reference herein to the term “myelofibrosis” includes any one of a disorder selected from primary myelofibrosis (PMF), post-essential thrombocythemia myelofibrosis (PET-MF) and post-polycythemia vera myelofibrosis (PPV-MF). Suitably, myelofibrosis is PMF.

In one aspect, the present invention provides an ERK1/2 inhibitor (e.g., Compound A) or a pharmaceutical acceptable salt thereof, for use in the treatment of a Philadelphia-chromosome negative myeloproliferative neoplasm.

In another aspect, the present invention provides an ERK1/2 inhibitor (e.g., Compound A) or a pharmaceutical acceptable salt thereof, for use in the treatment of polycythemia vera (PV).

In one further aspect, the present invention provides an ERK1/2 inhibitor (e.g., Compound A) or a pharmaceutical acceptable salt thereof, for use in the treatment of myelofibrosis (MF) in a patient. Alternatively, in one aspect, the present invention provides an ERK1/2 inhibitor (e.g., Compound A) or a pharmaceutical acceptable salt thereof, for use in the manufacture of a medicament for the treatment of myelofibrosis (MF) in a patient. Alternatively, in one aspect the present invention provides a method of treating myelofibrosis (MF) in a patient comprising the step of administering therapeutically effective amount of an ERK1/2 inhibitor (e.g., Compound A) or a pharmaceutical acceptable salt thereof, to said patient.

The term “primary myelofibrosis” (PMF), as used herein, is defined with reference to “The 2016 revision to the World Health Organization (WHO) classification of myeloid neoplasms and acute leukemia”, as published in Blood, 2016, 127:2391-2405. Primary myelofibrosis encompasses prefibrotic/early primary myelofibrosis (prePMF) and overt primary myelofibrosis (overt PMF). Diagnosis of prePMF requires meeting the following 3 major criteria, and at least 1 minor criterion according to the 2016 WHO classification for prePMF in table A:

TABLE A
Criteria for diagnosis of prePMF

Major criteria (prePMF)
1. Megakaryocytic proliferation and atypia, without reticulin fibrosis >grade 1, accompanied by
increased age-adjusted BM cellularity, granulocytic proliferation, and often decreased
erythropoiesis
2. Not meeting the WHO criteria for BCR-ABL1⁺CML, PV, ET, myelodysplastic syndromes, or
other myeloid neoplasms
3. Presence of JAK2, CALR, or MPL mutation or in the absence of these mutations, presence
of another clonal marker (e.g., ASXL1, EZH2, TET2, IDH1/IDH2, SRSF2, SF3B1) are of help
in determining the clonal nature of the disease or absence of minor reactive bone marrow
(BM) reticulin fibrosis (Minor (grade 1) reticulin fibrosis secondary to infection, autoimmune
disorder or other chronic inflammatory conditions, hairy cell leukemia or other lymphoid
neoplasm, metastatic malignancy, or toxic (chronic) myelopathies)
Minor criteria (prePMF)
Presence of at least 1 of the following, confirmed in 2 consecutive determinations:
a. Anemia not attributed to a comorbid condition
b. Leukocytosis ≥11*10⁹/L
c. Palpable splenomegaly
d. LDH increased to above upper normal limit of institutional reference range

Diagnosis of overt PMF requires meeting the following 3 major criteria, and at least 1 minor criterion according to the 2016 WHO classification for overt PMF in table B:

TABLE B
Criteria for diagnosis of overt PMF

Major criteria (overt PMF)
1. Presence of megakaryocytic proliferation and atypia, accompanied by either reticulin and/or
collagen fibrosis grades 2 or 3
2. Not meeting WHO criteria for ET, PV, BCR-ABL1⁺CML, myelodysplastic syndromes, or other
myeloid neoplasms
3. Presence of JAK2, CALR, or MPL mutation or in the absence of these mutations, presence of
another clonal marker (e.g., ASXL1, EZH2, TET2, IDH1/IDH2, SRSF2, SF3B1) or absence of
reactive myelofibrosis (BM fibrosis secondary to infection, autoimmune disorder, or other chronic
inflammatory conditions, hairy cell leukemia or other lymphoid neoplasm, metastatic malignancy,
or toxic (chronic) myelopathies)
Minor criteria (overt PMF)
Presence of at least 1 of the following, confirmed in 2 consecutive determinations:
a. Anemia not attributed to a comorbid condition
b. Leukocytosis ≥11*10⁹/L
c. Palpable splenomegaly
d. LDH increased to above upper normal limit of institutional reference range
e. Leukoerythroblastosis

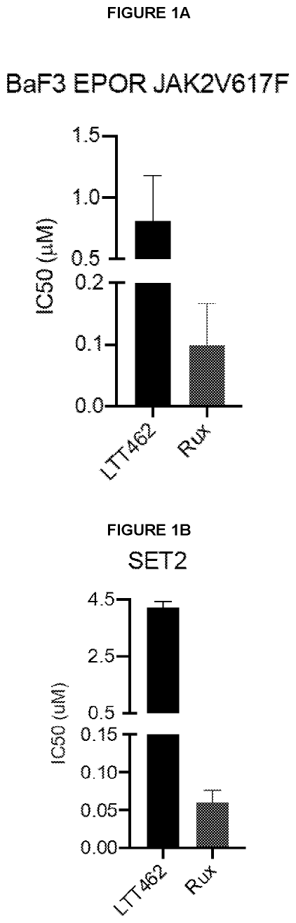

The term “bone marrow fibrosis”, as used herein, refers to bone marrow fibrosis graded according to the 2005 European consensus grading system (Thiele et. al., Haematologica, 2005, 90(8), 1128-1132, in particular as defined in Table 3 and FIG. 1 of page 1130 therein), such as:
“fibrosis grade 0”: scattered linear reticulin with no intersections (cross-overs) corresponding to normal bone marrow;
“fibrosis grade 1”: loose network of reticulin with many intersections, especially in perivascular areas;
“fibrosis grade 2”: diffuse and dense increase in reticulin with extensive intersections, occasionally with only focal bundles of collagen and/or focal osteosclerosis;
“fibrosis grade 3”: diffuse and dense increase in reticulin with extensive intersections with coarse bundles of collagen, often associated with significant osteosclerosis;

wherein the grading (i.e. grading of fiber density and quality) is made on the basis of bone marrow biopsy specimen assessment.

The term “essential thrombocythemia” (ET), as used herein, is defined with reference to “The 2016 revision to the World Health Organization (WHO) classification of myeloid neoplasms and acute leukemia”, as published in Blood, 2016, 127:2391-2405. The term “post-essential thrombocythemia myelofibrosis” (PET-MF), as used herein, refers to MF secondary to ET (i.e. MF arising as a progression of ET), wherein ET is as defined herein above. According to the IWG-MRT criteria (Barosi G et al, Leukemia (2008) 22, 437-438), criteria for diagnosing post-essential thrombocythemia myelofibrosis are:

TABLE C
Criteria for diagnosis of post-essential thrombocythemia myelofibrosis

Required criteria:
1. Documentation of a previous diagnosis of essential thrombocythemia as defined by the
WHO criteria
2. Bone marrow fibrosis grade 2-3
Additional criteria (two are required):
1. Anemia and a ≥2 mg/ml decrease from baseline hemoglobin level
2. A leukoerythroblastic peripheral blood picture
3. Increasing splenomegaly defined as either an increase in palpable splenomegaly of ≥5 cm
(distance of the tip of the spleen from the left costal margin) or the appearance of a newly
palpable splenomegaly
4. Increased lactate dehydrogenase (LDH) (above reference level)
5. Development of ≥1 of three constitutional symptoms: >10% weight loss in 6 months, night
sweats, unexplained fever (>37.5° C.)

The term “polycythemia vera” (PV), as used herein, is defined with reference to “The 2016 revision to the World Health Organization (WHO) classification of myeloid neoplasms and acute leukemia”, as published in Blood, 2016, 127:2391-2405. The term “post-polycythemia myelofibrosis” (PPV-MF), as used herein, refers to MF secondary to PV (i.e. MF arising as a progression of PV). According to the IWG-MRT criteria (Barosi G et al, Leukemia (2008) 22, 437-438), criteria for diagnosing post-polycythemia myelofibrosis are:

TABLE D
Criteria for diagnosis of post-polycythemia myelofibrosis

Required criteria:
1. Documentation of a previous diagnosis of polycythemia vera as defined by the WHO
criteria
2. Bone marrow fibrosis grade 2-3 (on 0-3 scale)
Additional criteria (two are required):
1. Anemia or sustained loss of requirement of either phlebotomy (in the absence of
cytoreductive therapy) or cytoreductive treatment for erythrocytosis
2. A leukoerythroblastic peripheral blood picture
3. Increasing splenomegaly defined as either an increase in palpable splenomegaly of ≥5 cm
(distance of the tip of the spleen from the left costal margin) or the appearance of a newly
palpable splenomegaly
4. Development of ≥1 of three constitutional symptoms: >10% weight loss in 6 months, night
sweats, unexplained fever (>37.5° C.)

As used herein, the following response criteria as defined by the International Working Group-Myeloproliferative Neoplasms Research and Treatment (IWG-MRT) and the European Leukemia Net (ELN) response criteria for MF (Tefferi et al, Blood 2013 122:1395-1398, which is incorporated by reference in its entirety) are used herein:

TABLE E
International Working Group-Myeloproliferative Neoplasms Research and Treatment
(IWG-MRT) and the European Leukemia Net (ELN) response criteria for myelofibrosis

| Response | Required criteria (for all response categories, benefit must last for ≥12 |
|---|---|
| categories | weeks to qualify as a response) |
| Complete | Bone marrow:* Age-adjusted normocellularity; <5% blasts; ≤grade 1 MF^{†} and |
| response | Peripheral blood: Hemoglobin ≥100 g/L and <UNL; neutrophil count ≥1 × |
| (CR) | 10⁹/L and <UNL; |
| | Platelet count ≥100 × 10⁹/L and <UNL; <2% immature myeloid cells^{‡} and |
| | Clinical: Resolution of disease symptoms; spleen and liver not palpable; no |
| | evidence of EMH |
| Partial | Peripheral blood: Hemoglobin ≥100 g/L and <UNL; neutrophil count ≥1 × 10⁹/L |
| response | and <UNL; platelet count ≥100 × 10⁹/L and <UNL; <2% immature myeloid |
| (PR) | cells^{‡} and |
| | Clinical: Resolution of disease symptoms; spleen and liver not palpable; no |
| | evidence of EMH or |
| | Bone marrow:* Age-adjusted normocellularity; <5% blasts; ≤grade 1 MF^{†} and |
| | peripheral blood: Hemoglobin ≥85 but <100 g/L and <UNL; neutrophil count |
| | ≥1 × 109/L and <UNL; platelet count ≥50, but <100 × 10⁹/L and <UNL; <2% |
| | immature myeloid cells^{‡} and |
| | Clinical: Resolution of disease symptoms; spleen and liver not palpable; no |
| | evidence of EMH |
| Clinical | The achievement of anemia, spleen or symptoms response without |
| improvement | progressive disease or increase in severity of anemia, thrombocytopenia, or |
| (CI) | neutropenia^{§} |
| Anemia | Transfusion-independent patients: a ≥20 g/L increase in hemoglobin level^{\|\|} |
| response | Transfusion-dependent patients: becoming transfusion-independent^{¶} |
| Spleen | A baseline splenomegaly that is palpable at 5-10 cm, below the LCM, |
| response^{#} | becomes not palpable** or |
| | A baseline splenomegaly that is palpable at >10 cm, below the LCM, |
| | decreases by ≥50%** |
| | A baseline splenomegaly that is palpable at <5 cm, below the LCM, is not |
| | eligible for spleen response |
| | A spleen response requires confirmation by MRI or computed tomography |
| | showing ≥35% spleen volume reduction |
| Symptoms | A ≥50% reduction in the MPN-SAF TSS^{††} |
| response | |

EMH, extramedullary hematopoiesis (no evidence of EMH implies the absence of pathology- or imaging study-proven nonhepatosplenic EMH); LCM, left costal margin; UNL, upper normal limit.
*Baseline and posttreatment bone marrow slides are to be interpreted at one sitting by a central review process.
^{†}Grading of MF is according to the European classification: Thiele et al. European consensus on grading bone marrow fibrosis and assessment of cellularity. Haematologica. 2005; 90:1128.
^{‡}Immature myeloid cells constitute blasts + promyelocytes + myelocytes + metamyelocytes + nucleated red blood cells. In splenectomized patients, <5% immature myeloid cells is allowed.
^{§}Increase in severity of anemia constitutes the occurrence of new transfusion dependency or a ≥20 g/L decrease in hemoglobin level from pretreatment baseline that lasts for at least 12 weeks. Increase in severity of thrombocytopenia or neutropenia is defined as a 2-grade decline, from pretreatment baseline, in platelet count or absolute neutrophil count, according to the Common Terminology Criteria for Adverse Events (CTCAE) version 4.0. In addition, assignment to Cl requires a minimum platelet count of ≥25 000 × 10(9)/L and absolute neutrophil count of ≥0.5 × 10(9)/L.
^{||}Applicable only to patients with baseline hemoglobin of <100 g/L. In patients not meeting the strict criteria for transfusion dependency at the time of treatment initiation, but have received transfusions within the previous month, the pre-transfusion hemoglobin level should be used as the baseline.
^{¶}Transfusion dependency is defined as transfusions of at least 6 units of packed red blood cells (PRBC), in the 12 weeks prior to start of treatment initiation, for a hemoglobin level of <85 g/L, in the absence of bleeding or treatment-induced anemia. In addition, the most recent transfusion episode must have occurred in the 28 days prior to start of treatment initiation. Response in transfusion-dependent patients requires absence of any PRBC transfusions during any consecutive “rolling” 12-week interval during the treatment phase, capped by a hemoglobin level of ≥85 g/L.
^{#}In splenectomized patients, palpable hepatomegaly is substituted with the same measurement strategy.
**Spleen or liver responses must be confirmed by imaging studies where a ≥35% reduction in spleen volume, as assessed by MRI or CT, is required. Furthermore, a ≥35% volume reduction in the spleen or liver, by MRI or CT, constitutes a response regardless of what is reported with physical examination.
^{††}Symptoms are evaluated by the MPN-SAF TSS. The MPN-SAF TSS is assessed by the patients themselves and this includes fatigue, concentration, early satiety, inactivity, night sweats, itching, bone pain, abdominal discomfort, weight loss, and fevers. Scoring is from 0 (absent/as good as it can be) to 10 (worst imaginable/as bad as it can be) for each item. The MPN-SAF TSS is the summation of all the individual scores (0-100 scale). Symptoms response requires ≥50% reduction in the MPN-SAF TSS.

In one embodiment the present invention provides an ERK1/2 inhibitor, suitably Compound A, for use in the treatment of myelofibrosis, especially primary MF, wherein the patient achieves complete response to the treatment according to the criteria in Table.

In one embodiment the present invention provides an ERK1/2 inhibitor, suitably Compound A, for use in the treatment of myelofibrosis, especially primary MF, wherein the patient achieves partial response to the treatment according to the criteria in Table.

Among patients, myelofibrosis frequently causes shortened survival due to disease transformation to acute leukemia, progression without acute transformation, cardiovascular complications or thrombosis, infection or portal hypertension. It is one of the aims of the present invention to improve the median survival of myelofibrosis patients.

As used herein, the term “median survival time” refers to the time of diagnosis or from the time of initiation of treatment according to the present invention that half of the patients in a group of patients diagnosed with the disease are still alive compared to patients receiving best available treatment or compared to patients receiving placebo and wherein patients belong to the same risk group of myelofibrosis, for example as described by Gangat et al (J Clin Oncol. 2011 Feb. 1; 29(4):392-397), which is hereby incorporated by reference in its entirety.

Accordingly, in one embodiment the present invention provides an ERK1/2 inhibitor, suitably Compound A, for use in the treatment of myelofibrosis, especially primary MF, wherein median survival time is increased by at least 3 months in the group of high risk MF patients or by at least six months, preferably by at least 12 months in the group of medium risk MF patients.

The compounds and the methods of the invention may be used to treat a patient as described herein.

As used herein, the term “patient” refers to a human being. In certain embodiments, the human patient is in need of treatment of a particular condition or disease. The compounds described herein are suitable for treating human patients having a disorder that can be treated by modulating (e.g., augmenting or inhibiting) an immune response.

The patient may be a patient suffering from myeloproliferative neoplasms (MPNs) such as myelofibrosis (MF), essential thrombocythemia (ET) and/or polycythemia vera (PV). For example, the patient may be suffering from PMF, PPV-MF, or PET-MF.

In certain embodiments, the patient, e.g. an adult patient, suffering from PMF, PPV-MF, or PET-MF.

In certain embodiments, the patient is a patient suffering from PMF, PPV-MF, or PET-MF and, in addition at baseline, shows one or more, or all characteristics selected from: (a) has Hb<11 g/dL (≤6.8 mmol/L); (b) is responsive and/or stable on treatment with a JAK inhibitor, such as ruxolitinib, and (c) exhibits measurable splenomegaly with spleen volume of ≥450 cm³

In certain embodiments, the human patient has a disorder described herein, e.g., myeloproliferative neoplasms (MPNs) such as myelofibrosis (MF), essential thrombocythemia (ET) and/or polycythemia vera (PV), is responsive and/or stable on treatment with a JAK inhibitor, such as ruxolitinib, and is in need for additional treatment options. In certain embodiments, the human patient has hemoglobin level less than 10 g/dL, a confirmed diagnosis of PMF, PPV-MF, or PET-MF, a palpable spleen of at least 5 cm from the left costal margin (LCM) and/or enlarged spleen volume of at least 450 cm³ per MRI or CT-scan, not suitable for, or not responsive and/or not stable on JAK inhibitor therapy such as ruxolitinib, and in need of additional treatment options. In certain embodiments, the human patients with PMF, PPV-MF, or PET-MF and receiving treatment with compounds described herein achieve a hemoglobin improvement of 2.0 g/dL or 1.5 g/dL from baseline, arrestment and/or improvement in spleen size, and/or improvement in bone marrow fibrosis of 1 grade from baseline.

The expression “is responsive and/or stable on treatment with a JAK inhibitor, such as ruxolitinib” means for example, being on ruxolitinib therapy for a period of time such as at least 12 weeks, with an unchanged ruxolitinib dose (e.g. in the range 5-25 mg BID) for the previous 4 weeks prior to first dose of treatment. It can also mean that such a patient is on ruxolitinib therapy for a period of time such as at least 24 weeks, with an unchanged ruxolitinib dose (e.g. in the range 5-25 mg BID) for the previous 8 weeks prior to first dose of treatment.

The term “treat”, “treating”, “treatment” or “therapy”, as used herein, means obtaining beneficial or desired results, for example, clinical results. Beneficial or desired results can include, but are not limited to, alleviation of one or more symptoms, as defined herein. One aspect of the treatment is, for example, that said treatment should have a minimal adverse effect on the patient, e.g. the agent used should have a high level of safety, for example without producing the side effects of a previously known therapy. The term “alleviation”, for example in reference to a symptom of a condition, as used herein, refers to reducing at least one of the frequency and amplitude of a symptom of a condition in a patient.

As used herein, the term “newly diagnosed” refers to diagnosis of the disorder, e.g. myelofibrosis and said patient has not received any treatment for the disorder. In one embodiment, the present invention provides an ERK1/2 inhibitor (e.g. Compound A) or a pharmaceutical acceptable salt thereof, for use in the treatment of a newly diagnosed myelofibrosis patient.

The term “triple-negative myelofibrosis patient”, as used herein, refers to a patient who lacks JAK2, CALR and MPL mutations. In one embodiment, the present invention provides an ERK1/2 inhibitor (e.g. Compound A) or a pharmaceutical acceptable salt thereof, for use in the treatment of triple-negative myelofibrosis patient.

The term “best available therapy”, as used herein, refers to any commercially available agent approved for example prior to March 2018, or prior to October 2020, for the treatment of PMF, PET-MF or PPV-MF, as monotherapy. Exemplary agents include, but are not limited to JAK inhibitors such as ruxolitinib or a pharmaceutically acceptable salt thereof, antineoplastic agents (e.g., hydroxyurea, anagrelide), glucocorticoids (e.g., prednisone/prednisolone, methylprednisolone), antianemia preparations (e.g., epoetin-alpha), immunomodulatory agents (e.g., thalidomide, lenalidomide), purine analogs (e.g., mercaptopurine, thioguanine), antigonadotropins (e.g., danazol), interferons (e.g., PEG-interferon-alpha 2a, interferon-alpha), nitrogen mustard analogs (e.g. melphalan), pyrimidine analogs (e.g., cytarabine).

The term “splenomegaly”, as used herein, refers to a palpably enlarged spleen (e.g. a spleen is palpable at ≥5 cm below the left coastal margin) or to an enlarged spleen as detected by an imaging test (e.g. a computed tomography (CT) scan, MRI, X-rays or ultrasound), wherein the term “enlarged spleen” refers to a spleen greater in size than normal (e.g., median normal spleen volume of 200 cm³).

The term “treatment of splenomegaly”, as used herein, refers to “improvement of splenomegaly”, which means a decrease in splenomegaly, for example a reduction in spleen volume, as defined by the International Working Group-Myeloproliferative Neoplasms Research and Treatment (IWG-MRT) and the European Leukemia Net (ELN) response criteria for MF in Table. In one embodiment, the invention may provide the use of an ERK1/2 inhibitor (e.g. Compound A) or a pharmaceutical acceptable salt thereof, for treatment of myelofibrosis, particularly for the treatment of splenomegaly associated with myelofibrosis, resulting in, for example, ≥20%, ≥25%, ≥30% or ≥35% reduction in spleen volume as measured by magnetic resonance imaging (MRI) or computed tomography (CT) from pre-treatment baseline to, for example, week 24 or week 48.

The term “hepatomegaly”, as used herein, refers to a palpably enlarged liver or to an enlarged liver as detected by an imaging test (e.g. a computed tomography (CT) scan), wherein the term “enlarged liver” refers to a liver greater in size than normal (e.g., median normal liver volume of approximately 1500 cm³).

The term “treatment of hepatomegaly”, as used herein, refers to “improvement of hepatomegaly”, which means a decrease in hepatomegaly, for example a reduction in hepatomegaly, as defined according to the International Working Group-Myeloproliferative Neoplasms Research and Treatment (IWG-MRT) and the European Leukemia Net (ELN) response criteria for MF in the preceding table. Accordingly, in one embodiment, the present invention provides the use of an ERK1/2 inhibitor (e.g. Compound A) or a pharmaceutical acceptable salt thereof, for treatment of myelofibrosis, particularly for the treatment of hepatomegaly associated with myelofibrosis, resulting in, for example, ≥20%, ≥25%, ≥30% or ≥35% reduction in liver volume as measured by magnetic resonance imaging (MRI) or computed tomography (CT) from pre-treatment baseline to, for example, week 24 or week 48.

The term “thrombocytopenia”, as used herein, refers to a platelet count, in blood specimen laboratory test, lower than normal, or less than 150,000/ml. The term “severity of thrombocytopenia”, as used herein, refers, for example, to specific grade 1-4 of thrombocytopenia according to CTCAE (version 4.03).

The term “treatment of thrombocytopenia”, as used herein, refers to “stabilizing thrombocytopenia” or “improving thrombocytopenia”, in comparison to the pre-treatment situation or in comparison to best available therapy or to placebo control. The term “stabilizing thrombocytopenia” refers, for example, to prevent an increase in the severity of thrombocytopenia, namely the platelet count remains stable. The term “improving thrombocytopenia” refers to alleviation of the severity of thrombocytopenia, namely increasing blood platelet count. In one embodiment, the invention provides an ERK1/2 inhibitor (e.g. Compound A) or a pharmaceutical acceptable salt thereof, for use in the treatment of myelofibrosis, particularly for the treatment of thrombocytopenia associated with myelofibrosis, resulting in stabilizing thrombocytopenia or improving thrombocytopenia from pre-treatment baseline to, for example, week 24 or week 48 of treatment.

The term “neutropenia”, as used herein, refers to an absolute neutrophil count (ANC), in blood specimen laboratory test, lower than normal value, or less than 1500/ml. The term “severity of neutropenia”, as used herein, refers, for example, to specific grade 1-4 of neutropenia according to CTCAE (version 4.03).

The term “treatment of neutropenia”, as used herein, refers to “stabilizing neutropenia” or “improving neutropenia”, for example, in comparison to the pre-treatment situation or in comparison to best available therapy or to placebo control. The term “stabilizing neutropenia” refers, for example, to prevent an increase in the severity of neutropenia. The term “improving neutropenia” refers, for example, to a decrease in the severity of neutropenia. In one embodiment, the invention provides an ERK1/2 inhibitor (e.g. Compound A) or a pharmaceutical acceptable salt thereof, for use in the treatment of myelofibrosis, particularly for the treatment of neutropenia associated with myelofibrosis, resulting in stabilizing neutropenia or improving neutropenia from pre-treatment baseline to, for example, week 24 or week 48 of treatment.

The term “anemia”, as used herein, refers to hemoglobin level, in blood specimen laboratory test, of less than 13.5 gram/100 ml in men and hemoglobin level of less than 12.0 gram/100 ml in women. The term “severity of anemia”, as used herein, refers, for example, to specific grade 1-4 of anemia according to CTCAE (version 4.03)].

The term “treatment of anemia”, as used herein, refers to “stabilizing anemia” or “improving anemia”, for example, in comparison to the pre-treatment situation or in comparison to best available therapy or to placebo control. The term “stabilizing anemia” refers, for example, to prevent an increase in the severity of anemia (e.g. preventing that a “transfusion-independent” patient becomes a “transfusion-dependent” patient or preventing anemia grade 2 becomes anemia grade 3). The term “improving anemia” refers to a decrease in the severity of anemia or an improvement in hemoglobin level. In one embodiment, the invention may provide the use of an ERK1/2 inhibitor (e.g. Compound A) or a pharmaceutical acceptable salt thereof, for treatment of myelofibrosis, particularly for the treatment of anemia associated with myelofibrosis, resulting in stabilizing anemia or improving anemia from pre-treatment baseline to, for example, week 24 or week 48 of treatment.

The term “treatment of bone marrow fibrosis associated with MF”, as used herein, means “stabilizing bone marrow fibrosis” or “improving bone marrow fibrosis”, for example, in comparison to the pre-treatment situation or in comparison to best available therapy or to placebo control. The term “stabilizing bone marrow fibrosis” refers, for example, to prevent increase in severity of bone marrow fibrosis. The term “improving bone marrow fibrosis” refers to a decrease in severity of bone marrow fibrosis, for example, from pre-treatment baseline, according to the 2005 European consensus grading system. In one embodiment, the invention may provide the use of an ERK1/2 inhibitor (e.g. Compound A) or a pharmaceutical acceptable salt thereof, for treatment of myelofibrosis, particularly for the treatment of bone marrow fibrosis associated with MF, resulting in stabilizing bone marrow fibrosis or improving bone marrow fibrosis from pre-treatment baseline to, for example, week 24 or week 48 of treatment.

The term “constitutional symptoms associated with myelofibrosis”, as used herein, refers to common debilitating chronic myelofibrosis symptoms, such as fever, pruritus (i.e. itching), abdominal pain/discomfort, weight loss, fatigue, inactivity, early satiety, night sweats or bone pain; for example, as described by Mughal et al (Int J Gen Med. 2014 Jan. 29; 7:89-101).

The term “treatment of constitutional symptoms associated with myelofibrosis”, as used herein, refers to “improvement of constitutional symptoms associated with myelofibrosis”, for example, in comparison to the pre-treatment situation or in comparison to best available therapy or to placebo control, for example, a reduction in total symptom score as measured by the modified myelofibrosis symptom assessment form version 2.0 diary (modified MFSAF v2.0) (Cancer 2011; 117:4869-77; N Engl J Med 2012; 366:799-807, the entire contents of which are incorporated herein by reference). In one embodiment, the invention may provide the use of an ERK1/2 inhibitor (e.g. Compound A) or a pharmaceutical acceptable salt thereof, for treatment of myelofibrosis, particularly for the treatment of constitutional symptoms associated with myelofibrosis, resulting in improvement of constitutional symptoms associated with myelofibrosis from pre-treatment baseline to, for example, week 24 or week 48 of treatment.

In another embodiment of any use of the invention, one or more of the constitutional symptoms associated with MF are alleviated (e.g. by eliminating or by reducing intensity, duration or frequency). In one embodiment, the reduction of constitutional symptoms is at least ≥20%, at least ≥30%, at least ≥40%, or at least ≥50% as assessed by the modified MFSAF v2.0 from pre-treatment baseline to, for example, week 24 or week 48.

In one embodiment of any use of the invention, the ERK1/2 inhibitor, suitably Compound A, is administered subsequently or prior to splenectomy or radiotherapy, such as splenic irradiation.

Monotherapy

In one aspect the present invention provides an ERK1/2 inhibitor, suitably Compound A, for use in the treatment of MF.

In one aspect, the present invention provides Compound A, or a pharmaceutical acceptable salt thereof, for use in the treatment of myelofibrosis, wherein Compound A, or a pharmaceutical acceptable salt thereof, administered in therapeutically effective amounts.

The term “therapeutically effective amount” refers to an amount of a drug or a therapeutic agent that will elicit the desired biological and/or medical response of a tissue, system or an animal (including man) that is being sought by a researcher or clinician.

Administration and Treatment Regimen

Compound A may be administered either QD (once a day) or BID (twice a day), preferably QD. Preferably, the total daily dose (TTD) of Compound A is from 100-300 mg, or from 150-200 mg, or from 200-300 mg, e.g may be selected from 50, 100, 150, 200, 250 and 300 mg, preferably administered QD.

In one embodiment, Compound A, or a pharmaceutically acceptable salt thereof, may be administered orally at a daily dose of 100 mg, 200 mg, or 300 mg, preferably once daily.

Compound A can be generally administered in a unit dosage of about 1-2000 mg of active ingredient for a patient of about 50-70 kg, or about 1-500 mg or about 1-250 mg or about 1-150 mg or about 0.5-100 mg, or about 1-50 mg of active ingredient. The unit dosage may be administered once or repeatedly during the same day, or during the week. More specifically, daily dose of between 45 mg and 600 mg, or between 100 mg and 450 mg, particularly between 150 mg and 300 mg, or between 200 mg and 300 mg, may be suitable.

In one embodiment, Compound A is prepared for administration via oral delivery, and may be used as its hydrochloride salt. In some embodiments, the compound or its HCl salt is simply encapsulated in a pharmaceutically acceptable container such as a hard or soft gelcap for oral administration.

All publications and patent applications mentioned in the specification are indicative of the level of skill of those skilled in the art to which the presently disclosed inventive concepts pertain. All publications and patent applications are herein incorporated by reference to the same extent as if each individual publication or patent application was specifically and individually indicated to be incorporated by reference.

References in this specification to “the invention” are intended to reflect embodiments of the several inventions disclosed in this specification, and should not be taken as unnecessarily limiting of the claimed subject matter.

### LIST OF ABBREVIATIONS

AE adverse event
AML Acute myeloid leukemia
ANC Absolute neutrophil count
ASCT allogeneic hematopoietic stem cell transplantation
AUC Area under curve
BID twice a day
BM bone marrow
C1D1 Cycle 1 Day 1 (and sequentially for other cycles and days, eg C1D2, C2D1 etc.)
CT computed tomography
CTCAE Common Terminology Criteria for Adverse Events
CYP cytochrome P-450
DDI Drug-drug interaction
DLT dose-limiting toxicity
ECG Electrocardiogram
EORTC European Organization for Research and Treatment of Cancer
ERK extracellular signal-regulated kinase
ET essential thrombocythemia
Hb hemoglobin
IV Intravenous
IWG-MRT International Working Group-Myeloproliferative Neoplasms Research and
Treatment
JAK Janus kinase
LCM left costal margin
MF myelofibrosis
MPN myeloproliferative neoplasm
MRI magnetic resonance imaging
PD pharmacodynamic(s)
PFS progression free survival
PK pharmacokinetic(s)
PLT platelets
PMF primary myelofibrosis
PRBC packed red blood cells
PV polycythemia vera
QD once a day
QLQ-C30 Quality of Life Questionnaire-Core 30
QoL quality of life
RBC red blood cell(s)
RP2D recommended phase 2 dose
RR Response rate
SAF symptom assessment form
STAT signal transducer and activator of transcription
TLS Tumor lysis syndrome
TSS total symptom score
WHO World Health Organization

The Examples below are set forth to aid in the understanding of the inventions but are not intended to, and should not be construed to, limit its scope in any way.

### EXAMPLES

### Example 1: Compound a (LTT462) and Ruxolitinib (Rux) in MPN Cell Lines

The antiproliferative activity of Compound A and ruxolitinib was tested in the human line SET2 carrying the Jak2V617F mutation and in the murine Ba/F3 cell line stably expressing erythropoietin receptor (EpoR) as well as either wild-type JAK2 or Jak2V617F. To assess anti-proliferative effects of inhibitors, cells were seeded at 10′000/200 ul with increasing inhibitor concentrations in triplicate. Proliferation was assessed at 48h using the CellTiter-Glo viability assay (Promega) and normalized to cell growth in medium with equivalent volume of DMSO. The concentration inhibiting proliferation by 50% (IC50) was determined with GraphPad Prism 8.0.

The IC50 results for ruxolitinib and Compound A in Ba/F3 JAK2V617F cells and SET2 cells are shown in FIGS. 1A and 1B, respectively.

### Example 2: Compound a (LTT462) and Ruxolitinib in MPN Mouse Models

The antiproliferative activity of Compound A and ruxolitinib was tested using a Jak2V617F knock-in mouse model. Primarily, a Jak2V617F knock-in mouse model reflecting a polycythemia vera phenotype was used (Mullally A et al, Cancer Cell 2010), which is characterized by Jak2V617F expression in hematopoietic tissues based on expression of Cre-recombinase under the control of the Vav or the Mx-1 promoter.

For treatment studies, bone marrow (BM) from primary Jak2V617F Vav-Cre CD45.2 mice was mixed 1:1 with Jak2 wild-type CD45.1 BM and transplanted into lethally irradiated CD45.1 recipients. Development of the MPN phenotype was confirmed by peripheral blood counts 2 months after BM transplantation. Mice were randomized to treatment groups according to blood counts and treated by oral gavage for 1-4 weeks.

As a model of MPLW515L mutant MPN, CD117-enriched (Miltenyi) Balb/c BM was transduced with retroviral supernatant containing MSCV-hMPLW515L-IRES-GFP and injected i.v. into lethally irradiated Balb/c recipients. Development of the MPN phenotype was confirmed by blood counts 2-4 weeks after BM transplantation. For treatment studies, mice were randomized to treatment groups according to blood counts and treated by oral gavage for 1-4 weeks. The suppression of ERK signaling was confirmed in splenocytes by Western blot analysis. For histopathology, tissues were fixed in 4% paraformaldehyde, paraffin-embedded and stained with hematoxylin/eosin. Gomori stain was used for assessment of reticulin fibers. Fibrosis was graded by a specialized hematopathologist. For flow cytometry analyses, BM cells were stained for lineage markers, Sca-1, c-Kit, CD41, CD150, CD48, CD16/32 and CD105, CD71 and Ter-119 (eBioscience) and for CD45.1 and CD45.2 alleles to assess mutant allele burden as the fraction of CD45.2+total BM or erythroid progenitor cells. Analyses were performed on a LSRFortessa (BD).

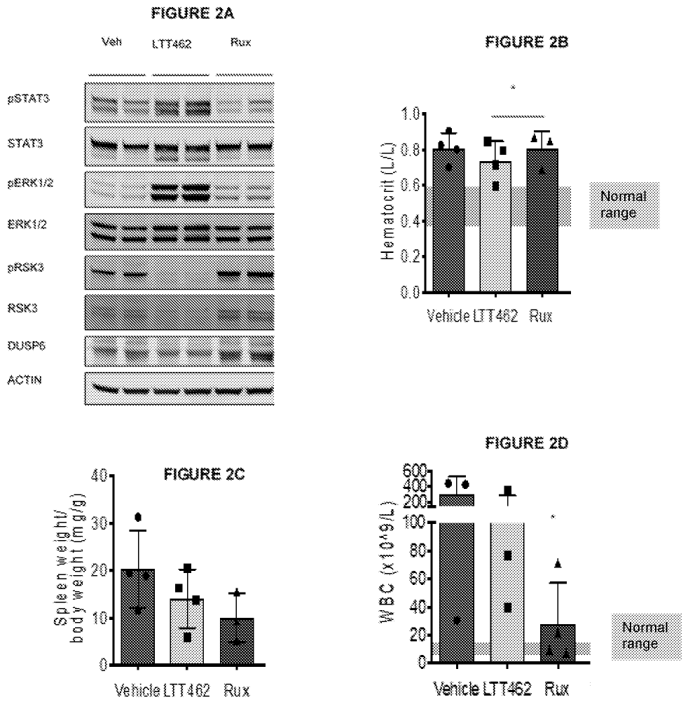

Compound A inhibited activation of ERK downstream targets RSK3 and DUSP6 in the splenocytes of Jak2V617F mice (FIG. 2A) and potently corrected splenomegaly (FIG. 2C) and polyglobulia or polycythemia (FIG. 2B). In MPLW515L mice, the correction of leukocytosis with ruxolitinib was superior to that of Compound A (FIG. 2D).

### Example 3: Dose Response of Compound a (LTT462) and Ruxolitinib in JAK2VF Mouse Model

Compound A and ruxolitinib both significantly normalized splenomegaly, polycythemia and hematocrit in JAK2V617F PV/MF mouse model.

Mice were administered an oral dose of either vehicle, ruxolitinib at 60 mg/kg BID and Compound A at 75 mg/kg QD for 14 consecutive days.

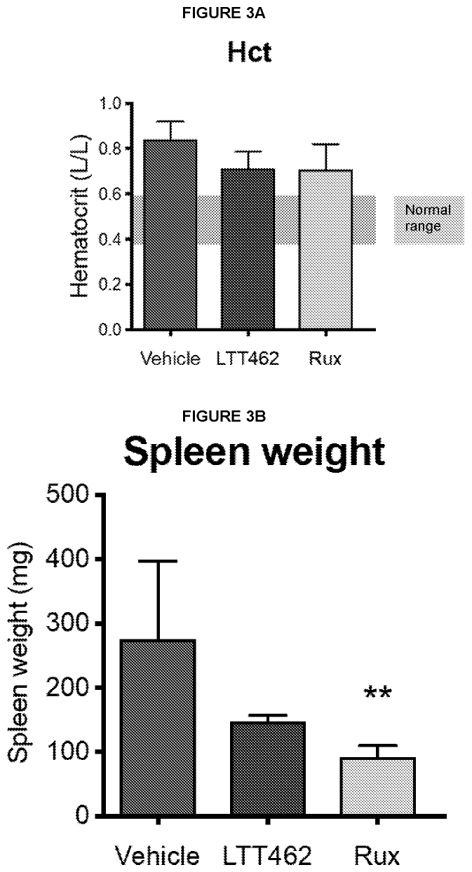

Ruxolitinib and Compound A significantly reduced elevated hematocrit (FIG. 3A) and splenomegaly (FIG. 3B) as compared to vehicle-treated mice.

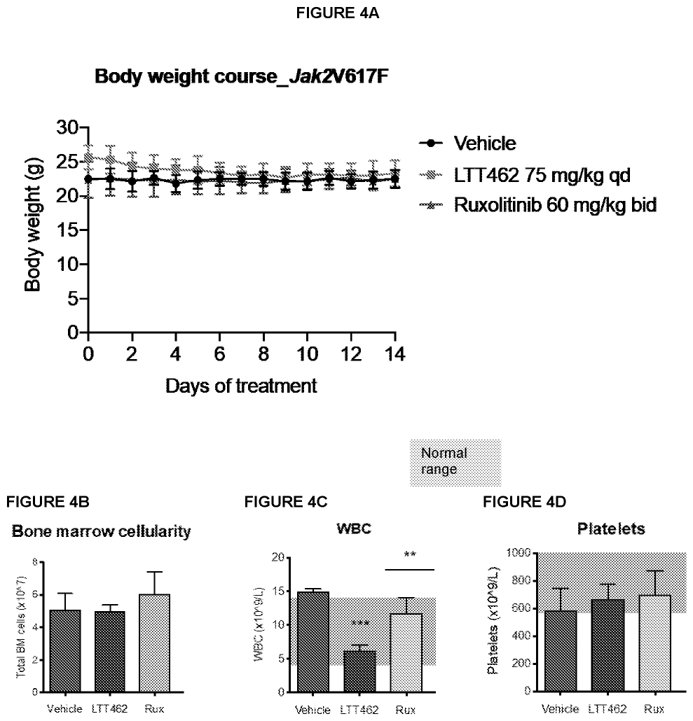

Furthermore, Compound A and ruxolitinib were both well tolerated as judged by lack of body weight loss (FIG. 4A), bone marrow cellularity (FIG. 4B), normal WBC count (FIG. 4C), and normal platelet count (FIG. 4D).

These results support the potential exploration of Compound A in polycythemia vera and myelofibrosis.

### Example 4: Compound a (LTT462) and Ruxolitinib in MPN Cells, JAK2V617F and MPLW5151L Mutant Mouse Models

Compound a (LTT462) and Ruxolitinib in CD34+Peripheral Blood Mononuclear Cells (PBMCs)

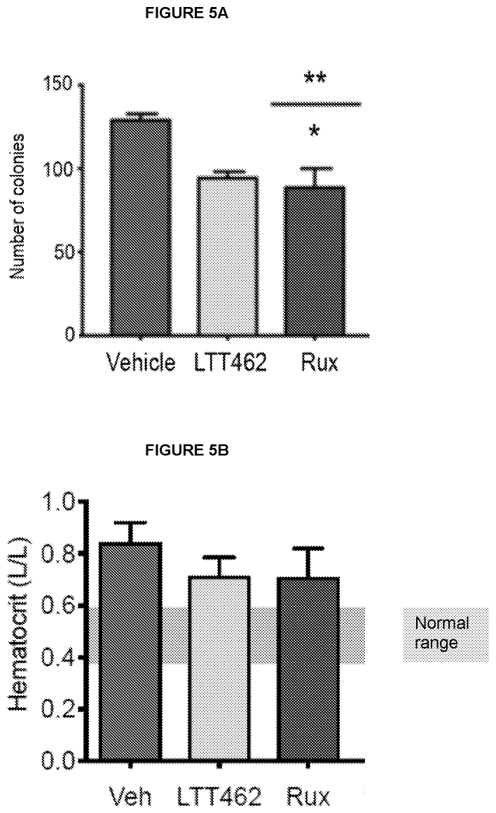

Ruxolitinib and Compound A comparably suppressed growth of the colonies derived from CD34+PBMCs of a MF patient (FIG. 5A).

Compound a (LTT462) and Ruxolitinib in Jak2V617F Mice

Ruxolitinib and Compound A resulted in comparable reduction of hematocrit (FIG. 5B).

Compound a (LTT462) and Ruxolitinib in EpoR Jak2V617F Mutant and Jak2 Wild Type Ba/F3 Cells

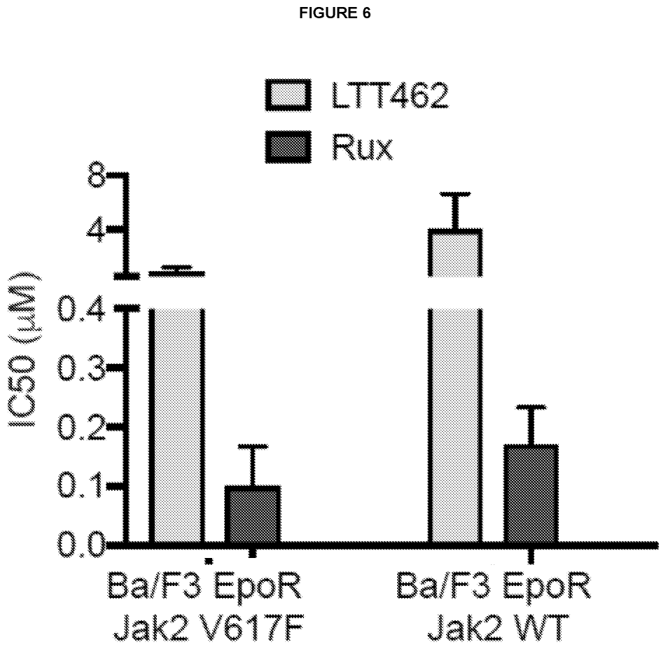

As seen by a stronger decrease in IC50 value, the suppression of proliferation in EpoR Jak2V617F mutant and Jak2 wild type Ba/F3 cells with ruxolitinib was superior to that of Compound A (FIG. 6).

These results support the potential exploration of Compound A in myeloproliferative neoplasms.

Dosing of Compound A

Compound A may be administered orally at a total daily dose of 100-300 mg, for example, 100 mg, 200 mg, or 300 mg, preferably administered once daily. Preferably Compound A is administered at a total daily dose of 100 mg or 200 mg, preferably administered once a day.

Thus in one embodiment, the present invention provides for the treatment of a patient suffering from an MPN, wherein the patient has been previously treated with ruxolitinib, or a pharmaceutically acceptable salt thereof.

In another embodiment, the present invention provides for the treatment of a patient suffering from an MPN, wherein the patient has a peripheral blood platelet count of less than or equal to 50,000/μL before treatment.

In another embodiment, the present invention provides for the treatment of a patient suffering from an MPN, wherein the patient has a peripheral blood platelet count of less than or equal to 75,000/μL before treatment.

1. A method of treating myeloproliferative neoplasm (MPN) in a patient, comprising administering an ERK1/2 inhibitor to the patient.

2. The method of claim 1 wherein the myeloproliferative neoplasm is selected from myelofibrosis (MF), essential thrombocythemia (ET), polycythemia vera (PV) and combinations thereof.

3. The method of claim 2, wherein the myelofibrosis comprises is primary myelofibrosis (PMF), post-essential thrombocythemia myelofibrosis (PET-MF) or post-polycythemia vera myelofibrosis (PPV-MF).

4. The method of claim 3, wherein said patient has thrombocytopenia associated with myelofibrosis.

5. The method of claim 3, wherein said patient has neutropenia associated with myelofibrosis.

6. The method of claim 3, wherein said patient has a peripheral blood platelet count of less than or equal to 50,000/μL before treatment.

7. The method of claim 3, wherein said patient has a peripheral blood platelet count of less than or equal to 75,000/μL before treatment.

8. The method of claim 1, wherein the myeloproliferative neoplasm (MPN) is primary myelofibrosis (PMF).

9. The method of claim 1 wherein median survival time increases by at least 3 months after treatment.

10. The method of claim 1 wherein said patient achieves an Hb improvement of ≥2.0 g/dL or 1.5 g/dL after treatment.

11. The method of claim 1 wherein said patient completely responds to the treatment.

12. The method of claim 1, wherein said myeloproliferative neoplasm (MPN) is newly diagnosed MF.

13. The method of to claim 1, wherein the patient is receiving or has received prior therapy with ruxolitinib.

14. The method of claim 13 wherein the prior therapy with ruxolitinib is administration at 5 mg twice daily, 10 mg twice daily, 15 mg twice daily, 20 mg twice daily or 25 mg twice daily.

15. The method of claim 1, wherein said ERK1/2 inhibitor is 4-(3-amino-6-((1S,3S,4S)-3-fluoro-4-hydroxycyclohexyl)pyrazin-2-yl)-N-((S)-1-(3-bromo-5-fluorophenyl)-2-(methylamino)ethyl)-2-fluorobenzamide (Compound A), or pharmaceutical acceptable salt thereof.

16. The method of claim 15 wherein Compound A is administered at a total daily dose of 100-300 mg, or 200-300 mg.

17. The method of claim 16 wherein Compound A is administered at a total daily dose of 100 mg or 200 mg, administered once a day.
